(19) United States
(12) Patent Application Publication
(10) Pub. No.: US 2022/0242766 A1
(43) Pub. Date: Aug. 4, 2022

(54) WASTEWATER TREATMENT APPARATUS AND WASTEWATER TREATMENT METHOD

(71) Applicants: KABUSHIKI KAISHA TOSHIBA, Tokyo (JP); TOSHIBA INFRASTRUCTURE SYSTEMS & SOLUTIONS CORPORATION, Kanagawa (JP)

(72) Inventors: Miwa SAITO, Meguro Tokyo (JP); Takumi OBARA, Fuchu Tokyo (JP); Jinyang HU, Inagi Tokyo (JP); Kenji KAKINUMA, Ota Tokyo (JP)

(21) Appl. No.: 17/579,062

(22) Filed: Jan. 19, 2022

(30) Foreign Application Priority Data
Feb. 1, 2021 (JP) 2021-014437

(51) Int. Cl. C02F 3/34; C02F 3/10; C02F 3/00; C02F 3/08; C12N 1/20

(52) U.S. Cl. CPC C02F 3/348; C02F 3/10; C02F 3/006; C02F 2101/308; C02F 3/085; C12N 1/20; C02F 3/082

(57) ABSTRACT

A wastewater treatment apparatus includes a treatment mechanism that treats a wastewater containing an organic chromaticity component with an enzyme produced by Bacillus proteolyticus.

### CROSS-REFERENCE TO RELATED APPLICATIONS

This application is based upon and claims the benefit of priority from Japanese Patent Application No. 2021-014437, filed on Feb. 1, 2021, and the entire contents of which are incorporated herein by reference.

### FIELD

Embodiments described herein relate generally to a wastewater treatment apparatus and a wastewater treatment method.

### BACKGROUND

Treatment of colored wastewater has become an issue in developing countries.

A pigment component contained in the colored wastewater is persistent organic matter etc. In addition, the colored wastewater containing the pigment component has a high chromaticity when visually confirmed even when the colored wastewater meets a discharge standard, and the colored wastewater is frequently mistaken for improperly treated wastewater. Therefore, a higher decomposition rate is required for the pigment component in the colored wastewater.

Further, for example, physicochemical treatment methods such as coagulation treatment, ozone treatment, and electrolytic treatment have been known.

However, these physicochemical treatment methods increase the running cost. For this reason, the methods are difficult to be adopted in developing countries.

On the other hand, biological treatment methods such as activated sludge treatment are widely adopted for normal organic wastewater treatment due to the relatively low cost.

However, it is difficult to decompose the persistent pigment component described above by the biological treatment method. In the persistent pigment component, azo dyes are known to be difficult to biodegrade.

### BRIEF DESCRIPTION OF THE DRAWINGS

FIG. 1 is a diagram illustrating an example of a configuration of a wastewater treatment apparatus according to a first embodiment.

FIG. 2 is a diagram illustrating an example of a configuration of a wastewater treatment apparatus according to a first modification of the first embodiment.

FIG. 3 is a diagram illustrating an example of a configuration of a wastewater treatment apparatus according to a second embodiment.

FIG. 4 is a diagram illustrating an example of a configuration of a wastewater treatment apparatus according to a modification of the second embodiment.

FIG. 5 is a graph illustrating a change over time in an absorbance of an Acid Red 88-containing liquid culture medium to which a Bacillus proteolyticus-containing culture solution is added according to an example.

FIG. 6 is a graph illustrating a change over time in a decrease rate of Acid Red 88 contained in a liquid culture medium to which Bacillus proteolyticus-containing culture solution is added according to an example.

### DETAILED DESCRIPTION

The present inventors have discovered a pigment decomposition ability as a new function of Bacillus proteolyticus belonging to the genus Bacillus. The embodiments described below relate to wastewater treatment utilizing the pigment decomposition ability of Bacillus proteolyticus. Chromaticity is an index showing the degree of color of water. In the following description, the phrase wastewater containing an organic chromaticity component may be referred to as colored wastewater. Also, the word pigment may be referred to as coloring matter.

### First Embodiment

The first embodiment will be described with reference to the drawings.

### (Configuration Example of Wastewater Treatment Apparatus)

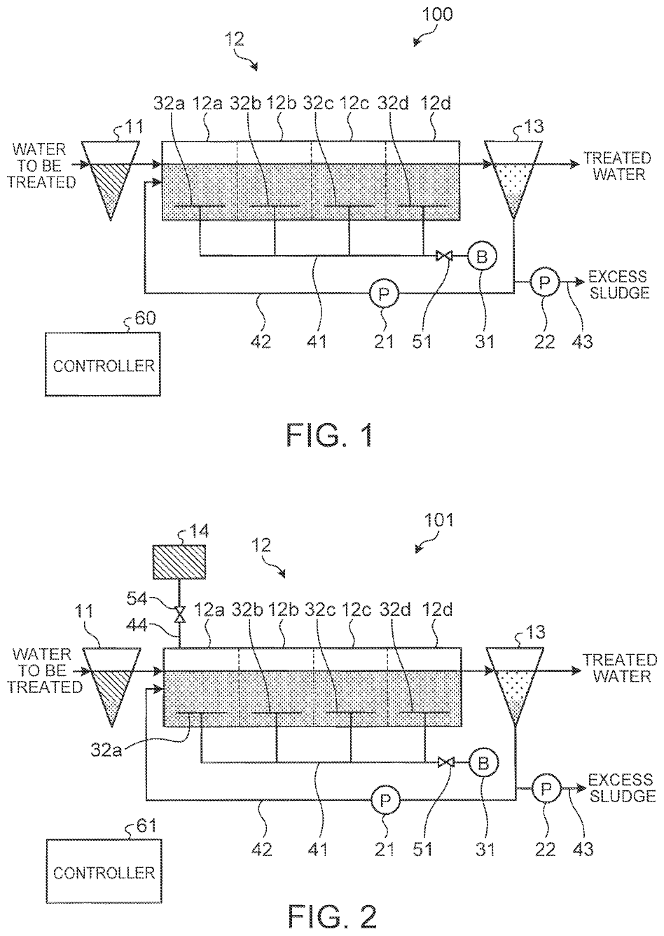

FIG. 1 is a diagram illustrating an example of a configuration of a wastewater treatment apparatus 100 according to a first embodiment. The wastewater treatment apparatus 100 treats wastewater which is water to be treated and discharges the treated water to the outside of the wastewater treatment apparatus 100. The wastewater to be treated by the wastewater treatment apparatus 100 is, for example, colored wastewater containing an organic chromaticity component. The organic chromaticity component is, for example, an azo dye containing an azo bond (—N═N—).

As illustrated in FIG. 1, the wastewater treatment apparatus 100 of the first embodiment includes a first settling basin 11, a reaction tank 12, a final settling basin 13, and a controller 60 (control unit). In FIG. 1, the first settling basin 11, the reaction tank 12, and the final settling basin 13 are horizontally disposed. However, the first settling basin 11, the reaction tank 12, and the final settling basin 13 may be disposed to have a gradient so that arrangement positions are lowered in this order. In this way, the water to be treated can naturally flow down from the first settling basin 11 on an upstream side to the final settling basin 13 on a downstream side.

The first settling basin 11 temporarily stores the water to be treated flowing into the wastewater treatment apparatus 100. In this way, a suspended material, which has a relatively high specific gravity, contained in the water to be treated can be deposited in the first settling basin 11 as sludge etc. and separated from the water to be treated. Note that at a bottom of the first settling basin 11, a sludge discharge pipe (not illustrated) for discharging the deposited sludge is provided.

Supernatant water of the water to be treated from which a suspended material is separated is sent to the reaction tank 12 in a subsequent stage.

The reaction tank 12 is a water tank having a mechanism for decomposing and removing the organic chromaticity component in the water to be treated by an action of Bacillus proteolyticus. The reaction tank 12 includes, for example, a plurality of compartments 12a to 12d. These compartments 12a, 12b, 12c, and 12d are disposed in this order from the upstream side to the downstream side. However, the number of compartments 12a to 12d provided in the reaction tank 12 is arbitrary.

Note that in the configuration of the first embodiment, the reaction tank 12 is included in a treatment mechanism for treating wastewater by Bacillus proteolyticus. Aeration equipment (blower 31, air supply pipe 41, valve 51, and air diffuser plate 32) and return equipment (return pipe 42 and pump 21) described later may be included in the treatment mechanism.

The wastewater treatment apparatus 100 is provided with the aeration equipment that aerates the water to be treated in the reaction tank 12. The aeration equipment includes the blower 31, the air supply pipe 41, the valve 51, and a plurality of air diffuser plates 32 (32a to 32d).

The blower 31 sends a gas such as air to the air diffuser plates 32 through the air supply pipe 41. One end of the air supply pipe 41 is connected to the blower 31. The other end of the air supply pipe 41 is branched into a plurality of parts and is connected to each of the plurality of air diffuser plates 32. The plurality of air diffuser plates 32a to 32d is disposed in the plurality of compartments 12a to 12d of the reaction tank 12, respectively. A valve 51 is provided in the air supply pipe 41, and by opening and closing the valve 51, gas delivery from the blower 31 to the air diffuser plates 32 is started and stopped.

By such an aeration equipment, the gas from the blower 31 is supplied to each of the compartments 12a to 12d. By supplying a gas to the reaction tank 12 injected with Bacillus proteolyticus, oxygen contained in the gas such as air promotes a reaction between an enzyme of Bacillus proteolyticus and the organic chromaticity component.

The final settling basin 13 temporarily stores the water to be treated flowing out of the reaction tank 12. In this way, the suspended material remaining in the water to be treated can be deposited as sludge etc. and separated from the water to be treated. The sludge discharge pipe 43 is provided at a bottom of the final settling basin 13. A pump 22 is provided in the sludge discharge pipe 43, and excess sludge deposited at the bottom of the final settling basin 13 is discharged to the outside of the wastewater treatment apparatus 100.

The final settling basin 13 is provided with the return equipment for returning a part of the water to be treated to the reaction tank 12. The return equipment includes the return pipe 42 and the pump 21. One end of the return pipe 42 is connected to the final settling basin 13, and the other end is connected to, for example, the uppermost stream compartment 12a of the reaction tank 12. The pump 21 is provided in the return pipe 42.

By such return equipment, a part of the water to be treated stored in the final settling basin 13 is returned to an upstream part of the reaction tank 12 and treated a plurality of times. Note that the sludge discharge pipe 43 described above is connected to the return pipe 42. In this way, mixing of excess sludge into the water to be treated returned to the reaction tank 12 is suppressed.

The supernatant water of the water to be treated from which the remaining suspended material is separated in the final settling basin 13 is discharged from the final settling basin 13 as treated water.

Disinfection equipment (not illustrated) is provided on the downstream side of the final settling basin 13. The treated water discharged from the final settling basin 13 is disinfected in the disinfection equipment and discharged into a river or the ocean.

The controller 60 is configured as a computer including, for example, a central processing unit (CPU), a read only memory (ROM), a random access memory (RAM), etc., and controls each part of the wastewater treatment apparatus 100. That is, the controller 60 controls the start and stop of the operation of the blower 31 and the pumps 21 and 22, and controls the opening and closing of the valve 51.

Further, the controller 60 may receive a signal from a sensor (not illustrated) etc. provided in the first settling basin 11 and monitor the concentration of the organic chromaticity component contained in the water to be treated stored in the first settling basin 11. Further, the controller 60 may receive a signal from a sensor (not illustrated) etc. provided in the final settling basin 13 and monitor the concentration of the organic chromaticity component contained in the water to be treated stored in the final settling basin 13. Further, the controller 60 may receive a signal from a sensor (not illustrated) etc. provided in the reaction tank 12 and monitor the concentration of the organic chromaticity component contained in the water to be treated during treatment in the reaction tank 12. As a sensor capable of detecting the concentration of the organic chromaticity component of the water to be treated, for example, it is possible to use an absorption photometer etc. that measures the absorbance of the water to be treated at a predetermined wavelength.

When the controller 60 controls the pump 21 etc. based on the concentration of the organic chromaticity component of the water to be treated in at least one of the first settling basin 11, the reaction tank 12, and the final settling basin 13, it is possible to adjust the amount of water to be treated returned from the final settling basin 13 to the reaction tank 12.

Specifically, the controller 60 may increase the amount of the water to be treated returned from the final settling basin 13 to the reaction tank 12 when the concentration of the organic chromaticity component of the water to be treated is higher than a predetermined value. In addition, the control unit 60 may decrease the amount of the water to be treated returned from the final settling basin 13 to the reaction tank 12 when the concentration of the organic chromaticity component of the water to be treated is lower than the predetermined value.

### (Example of Wastewater Treatment by Wastewater Treatment Apparatus)

Next, an example of wastewater treatment by the wastewater treatment apparatus 100 of the first embodiment will be described with reference to FIG. 1. As described above, in the wastewater treatment apparatus 100 of the first embodiment, Bacillus proteolyticus is used for the wastewater treatment.

When the wastewater treatment apparatus 100 is started, for example, inoculum (seed fungus) of Bacillus proteolyticus is injected into the reaction tank 12. Then, water containing the organic chromaticity component is poured into the reaction tank 12 injected with the inoculum of Bacillus proteolyticus to perform acclimation of the inoculum.

When the wastewater treatment apparatus 100 is in operation, the water to be treated is treated by Bacillus proteolyticus acclimatized to the organic chromaticity component. According to the newly discovered findings by the present inventors, Bacillus proteolyticus produces an enzyme having a decomposition ability of the organic chromaticity component. Specifically, this enzyme has a function of cleaving (cutting) an azo bond possessed by an azo dye etc. which is the organic chromaticity component. The function of this enzyme produced by Bacillus proteolyticus decomposes the organic chromaticity component contained in the water to be treated, and treats the water to be treated.

Bacillus proteolyticus is known to produce protease. Protease is an enzyme that cleaves peptide bond (—C(═O)—NH—) of protein. Therefore, in the reaction tank 12 injected with Bacillus proteolyticus, it is possible to decompose organic matter such as protein that may be contained in the water to be treated.

As mentioned above, Bacillus proteolyticus is known as a protease-producing microorganism. Protease produced by Bacillus proteolyticus cleaves the peptide bond of protein.

The inventors found that Bacillus proteolyticus produces an enzyme different from protease. Further, the enzyme has a function of cleaving the azo bond. As a result of diligent research, the inventors have succeeded in applying Bacillus proteolyticus to the treatment of the colored wastewater, which is a serious problem in developing countries, by utilizing the pigment decomposition ability of the enzyme.

According to the wastewater treatment apparatus 100 of the first embodiment, the reaction tank 12 for treating wastewater containing the organic chromaticity component using the enzyme produced by Bacillus proteolyticus is provided. In this way, for example, the presence of Bacillus proteolyticus in a biological treatment system enables treatment of the colored wastewater at a lower cost when compared to the physicochemical treatment. In this way, for example, it is possible to contribute to solving the problem of wastewater treatment in developing countries.

The embodiment provides a wastewater treatment apparatus that treats wastewater containing an organic chromaticity component. The wastewater treatment apparatus includes a treatment mechanism that treats the wastewater using an enzyme produced by Bacillus proteolyticus.

The embodiment provides a wastewater treatment apparatus and a wastewater treatment method capable of treating colored wastewater by low-cost biological treatment.

According to the wastewater treatment apparatus 100 of the first embodiment, since Bacillus proteolyticus also produces a protease, it is possible to decompose organic matter other than the organic chromaticity component. By this, it is unnecessary to separately provide a reaction tank or an apparatus, and a new operation such as a separate reaction is not required, so that the cost can be further suppressed.

### First Modification

Next, a wastewater treatment apparatus 101 of a first modification of the first embodiment will be described with reference to FIG. 2. The wastewater treatment apparatus 101 of the first modification is different from the wastewater treatment apparatus 100 of the first embodiment in that a culture tank 14 is provided.

FIG. 2 is a diagram illustrating an example of a configuration of the wastewater treatment apparatus 101 according to the first modification of the first embodiment. In FIG. 2, the same reference symbols are given to the same configurations as those of the above-described first embodiment, and a description thereof will be omitted.

As illustrated in FIG. 2, the wastewater treatment apparatus 101 of the first modification includes a culture tank 14, an injection pipe 44, and a valve 54 in addition to the respective configurations of the first embodiment. Further, the wastewater treatment apparatus 101 includes a control unit 61 instead of the control unit 60 of the first embodiment.

The culture tank 14 is disposed above the reaction tank 12, for example. The inoculum of Bacillus proteolyticus is injected into the culture tank 14, and the inoculum of Bacillus proteolyticus can be multiplied in the culture tank 14.

Specifically, the culture tank 14 is injected with a liquid culture medium for multiplying Bacillus proteolyticus. Further, the culture tank 14 includes, for example, a heater (not illustrated) for heating the liquid culture medium to a predetermined temperature. As the liquid culture medium, for example, it is possible to use a mixed solution containing components such as nutrient broth, glucose, sodium chloride, and soluble starch.

An azo dye such as Acid Red 88 may be further added to the mixed solution. By containing the azo dye in the liquid medium, it is possible to suppress the multiplication of microorganisms other than Bacillus proteolyticus in the culture tank 14. Further, when the liquid culture medium contains the azo dye, the effect of acclimatizing Bacillus proteolyticus to the azo dye can be expected.

For example, by holding the liquid culture medium at a temperature suitable for multiplication of Bacillus proteolyticus around 30° C. for several days, Bacillus proteolyticus can be multiplied in the culture tank 14.

One end of the injection pipe 44 is connected to a lower end of the culture tank 14. The other end of the injection pipe 44 is connected to the reaction tank 12. In this way, the culture tank 14 is connected to the reaction tank 12 of the wastewater treatment apparatus 101. It is preferable that the culture tank 14 is connected to the upstream side of the reaction tank 12, for example, a compartment 12a on the uppermost stream side of the reaction tank 12. A valve 54 is provided in the injection pipe 44. By opening and closing the valve 54, the inflow of the culture solution (culture liquid) containing Bacillus proteolyticus from the culture tank 14 to the reaction tank 12 is started and stopped.

Note that in the configuration of the first modification, the reaction tank 12, the culture tank 14, the injection pipe 44, and the valve 54 are included in a treatment mechanism for treating wastewater by Bacillus proteolyticus. The aeration equipment (blower 31, air supply pipe 41, valve 51, and air diffuser plate 32) and return equipment (return pipe 42 and pump 21) may be included in the treatment mechanism.

The controller 61 is configured as a computer including, for example, a CPU, ROM, RAM, etc., similarly to the controller 60 of the first embodiment described above, and controls each part of the wastewater treatment apparatus 101, including temperature adjustment and heat retention of the culture tank 14 and opening/closing of the valve 54.

The controller 61 adjusts the temperature of the culture tank 14 to an appropriate temperature and holds the culture tank 14 for a predetermined time, thereby multiplying Bacillus proteolyticus in the culture tank 14. Further, the controller 61 controls the opening and closing of the valve 54 of the injection pipe 44, thereby controlling at least one of an injection timing and the amount of injection of the culture solution containing Bacillus proteolyticus from the culture tank 14 to the reaction tank 12.

As described above, the control unit 61 may monitor the concentration of the organic chromaticity component of the water to be treated in at least one of the first settling basin 11, the reaction tank 12, and the final settling basin 13. The control unit 61 may control at least one of an injection timing and the amount of injection of the culture solution containing Bacillus proteolyticus from the culture tank 14 to the reaction tank 12 according to the concentration of the organic chromaticity component of the water to be treated.

Specifically, when the concentration of the organic chromaticity component of the water to be treated is higher than a predetermined value, the control unit 60 may increase a frequency of injection of the culture solution containing Bacillus proteolyticus from the culture tank 14 to the reaction tank 12 or increase the amount of injection. In addition, when the concentration of the organic chromaticity component of the water to be treated is lower than the predetermined value, the control unit 60 may decrease a frequency of injection of the culture solution containing Bacillus proteolyticus from the culture tank 14 to the reaction tank 12 or decrease the amount of injection.

According to the wastewater treatment apparatus 101 of the first modification, similar effect as that of the wastewater treatment apparatus 100 of the first embodiment is obtained.

According to the wastewater treatment apparatus 101 of the first modification, the culture tank 14 connected to the reaction tank 12 is provided. By this, it is possible to stably supply Bacillus proteolyticus to the reaction tank 12, and to stably perform pigment decomposition.

According to the wastewater treatment apparatus 101 of the first modification, the control unit 61 controls at least one of an injection timing and the amount of injection of Bacillus proteolyticus from the culture tank 14 to the reaction tank 12. By this, it is possible to omit a manual operation, reduce labor costs, and suppress human error.

### Second Modification

Next, a wastewater treatment apparatus of a second modification of the first embodiment will be described. The wastewater treatment apparatus of the second modification is different from the wastewater treatment apparatus 101 of the first modification in that a culture solution tank is included instead of the culture tank 14.

The culture solution tank of the second modification is connected to, for example, the upstream side of the reaction tank 12 by the same equipment as the injection pipe 44, the valve 54, etc. of the first modification described above. The culture solution in which Bacillus proteolyticus is cultured can be stored in the culture solution tank. The culture solution is a supernatant (supernatant liquid) etc. in which Bacillus proteolyticus is separated from the culture solution after culturing Bacillus proteolyticus by centrifugation etc. For this reason, the supernatant is virtually free of Bacillus proteolyticus. However, the supernatant contains a predetermined amount of enzyme having the pigment decomposition ability produced by Bacillus proteolyticus during a multiplication process. In order to increase the concentration of the enzyme, a concentrate (concentrated liquid) in which the supernatant is concentrated may be stored in the culture solution tank.

Note that in the configuration of the second modification, equipment such as the reaction tank 12, the culture solution tank, and the injection pipe and the valve connected to the culture solution tank is included in a treatment mechanism for treating wastewater by Bacillus proteolyticus. The aeration equipment (blower 31, air supply pipe 41, valve 51, and air diffuser plate 32) and return equipment (return pipe 42 and pump 21) may be included in the treatment mechanism.

Similarly to the controller 60 of the first embodiment, the controller of the second modification is configured as a computer including, for example, a CPU, ROM, RAM, etc., and controls each configuration of the wastewater treatment apparatus of the second modification, including control of at least one of a supply timing and the amount of supply of the culture solution from the culture solution tank to the reaction tank 12, specifically, the supernatant of the culture solution or the concentrate of the supernatant.

As described above, the controller of the second modification may monitor the concentration of the organic chromaticity component of the water to be treated in at least one of the first settling basin 11, the reaction tank 12, and the final settling basin 13. The controller of the second modification may control at least one of a supply timing and the amount of supply of the supernatant or the concentrate from the culture solution tank to the reaction tank 12 according to the concentration of the organic chromaticity component of the water to be treated.

Specifically, when the concentration of the organic chromaticity component of the water to be treated is higher than a predetermined value, the controller of the second modification may increase a frequency of supply of the supernatant or the concentrate from the culture solution tank to the reaction tank 12 or increase the amount of supply thereof. In addition, when the concentration of the organic chromaticity component of the water to be treated is lower than the predetermined value, the controller of the second modification may decrease the frequency of supply of the supernatant or the concentrate from the culture solution tank to the reaction tank 12 or decrease the amount of supply thereof.

According to the wastewater treatment apparatus of the second modification, similar effect as that of the wastewater treatment apparatus 100 of the first embodiment is obtained.

According to the wastewater treatment apparatus of the second modification, the culture solution tank connected to the reaction tank 12 is provided. In this way, an enzyme component having the pigment decomposition ability can be directly supplied to the reaction tank 12, so that the concentration of the enzyme in the reaction tank 12 etc. can be controlled more precisely. In addition, it is possible to suppress the consumption of Bacillus proteolyticus in the reaction tank 12, and it is possible to cause Bacillus proteolyticus separated from the culture solution to produce further enzymes.

### Third Modification

Next, a wastewater treatment apparatus of a third modification of the first embodiment will be described. The wastewater treatment apparatus of the third modification is different from the wastewater treatment apparatus 101 of the first modification in that a microbial formulation container (microbial or microorganism product container) is provided instead of the culture tank 14.

The microbial formulation container of the third modification is connected to, for example, the upstream side of the reaction tank 12 by the same equipment as the injection pipe 44, the valve 54, etc. of the first modification described above. The microbial formulation container can contain a microbial formulation containing Bacillus proteolyticus. The microbial formulation (microbial or microorganism product) can take various forms such as powder, liquid, or tablet.

Note that in the configuration of the third modification, equipment such as the reaction tank 12, the microbial formulation container, an injection pipe connected to the microbial formulation container, and the valve is included in a treatment mechanism for treating wastewater by Bacillus proteolyticus. The aeration equipment (blower 31, air supply pipe 41, valve 51, and air diffuser plate 32) and return equipment (return pipe 42 and pump 21) may be included in the treatment mechanism.

Similarly to the controller 60 of the first embodiment, the controller of the third modification is configured as a computer including, for example, a CPU, ROM, RAM, etc., and controls each configuration of the wastewater treatment apparatus of the third modification, including control of at least one of a supply timing and the amount of supply of the microbial formulation from the microbial formulation container to the reaction tank 12.

As described above, the controller of the third modification may monitor the concentration of the organic chromaticity component of the water to be treated in at least one of the first settling basin 11, the reaction tank 12, and the final settling basin 13. The controller of the third modification may control at least one of a supply timing and the amount of supply of the microbial formulation from the microbial formulation container to the reaction tank 12 according to the concentration of the organic chromaticity component of the water to be treated.

Specifically, when the concentration of the organic chromaticity component of the water to be treated is higher than a predetermined value, the controller of the third modification may increase a frequency of supply of the microbial formulation from the microbial formulation container to the reaction tank 12 or increase the amount of supply thereof. In addition, when the concentration of the organic chromaticity component of the water to be treated is lower than the predetermined value, the controller of the third modification may decrease the frequency of supply of the microbial formulation from the microbial formulation container to the reaction tank 12 or decrease the amount of supply thereof.

According to the wastewater treatment apparatus of the third modification, similar effect as that of the wastewater treatment apparatus 100 of the first embodiment is obtained.

According to the wastewater treatment apparatus of the third modification, the microbial formulation container connected to the reaction tank 12 is provided. By this, the microbial formulation containing Bacillus proteolyticus can be supplied to the reaction tank 12, so that the concentration of the enzyme in the reaction tank 12 etc. can be controlled more precisely. In addition, when Bacillus proteolyticus is in the form of the microbial formulation, handling becomes easy. In addition, it is easy to use in combination with a microbial formulation containing useful microorganisms other than Bacillus proteolyticus, and the wastewater treatment capacity can be further increased.

### Second Embodiment

The second embodiment will be described with reference to the drawings. The wastewater treatment apparatus of the second embodiment is different from the wastewater treatment apparatus 100 of the first embodiment in that an immobilized carrier (fixed carrier) is disposed on the upstream side of the reaction tank.

### (Configuration Example of Wastewater Treatment Apparatus)

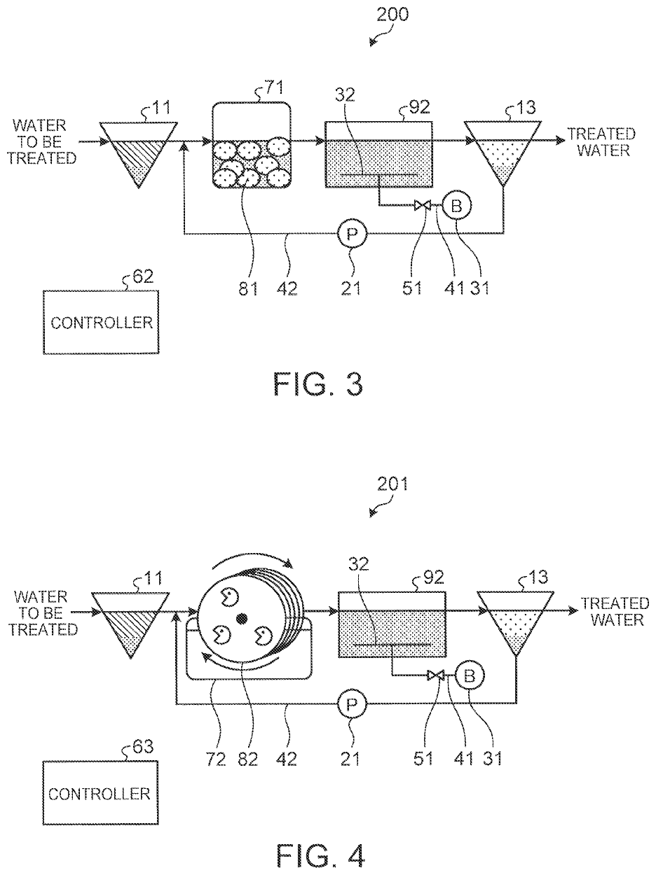

FIG. 3 is a diagram illustrating an example of a configuration of a wastewater treatment apparatus 200 according to the second embodiment. In FIG. 3, the same reference symbols are given to the same configurations as those of the above-described first embodiment, and a description thereof will be omitted.

As illustrated in FIG. 3, the wastewater treatment apparatus 200 of the second embodiment includes a first settling basin 11, a carrier immersion tank 71, a reaction tank 92, and a final settling basin 13 in this order from the upstream side. The first settling basin 11, the carrier immersion tank 71, the reaction tank 92, and the final settling basin 13 may be disposed to have a gradient so that arrangement positions are lowered in this order.

The carrier immersion tank 71 is disposed in a flow path of the water to be treated flowing from the first settling basin 11 to the reaction tank 92, and is configured, for example, so that the immobilized carrier 81 on which Bacillus proteolyticus is immobilized can be immersed in the water to be treated in the carrier immersion tank 71. In addition, for example, the carrier immersion tank 71 may include a heater (not illustrated) that heats the inside of the carrier immersion tank 71 to a temperature of about 30° C. suitable for the activity of Bacillus proteolyticus.

The immobilized carrier 81 is configured such that, for example, Bacillus proteolyticus can be dominantly attached and immobilized. That is, it is preferable that Bacillus proteolyticus is the dominant species in the immobilized carrier 81.

Specifically, the immobilized carrier 81 includes a material having a large specific surface area such as a porous material, or has a material having a large specific surface area on the outermost surface. The immobilized carrier 81 may have a shape such as a corrugated plate shape, a net shape, a string shape, a spherical shape, a cylindrical shape, or a honeycomb shape, for increasing the specific surface area.

When the immobilized carrier 81 has a large specific surface area, more Bacillus proteolyticus can be attached to and immobilized on the immobilized carrier 81. In addition, the contact efficiency between the immobilized carrier 81 and the water to be treated can be increased.

Further, the immobilized carrier 81 having the above-mentioned shape may be a fluidized bed carrier configured to be able to flow in the water to be treated in the carrier immersion tank 71. When the immobilized carrier 81 flows in the water to be treated, the contact efficiency between the immobilized carrier 81 and the water to be treated can be further increased.

In the carrier immersion tank 71, the enzyme produced by Bacillus proteolyticus is eluted in the water to be treated in contact with the immobilized carrier 81. The water to be treated is sent to the reaction tank 92 in a subsequent stage in a state where the enzyme having this pigment decomposition ability is contained.

The reaction tank 92 is a water tank having a mechanism for decomposing and removing the organic chromaticity component by causing a reaction between the enzyme and the organic chromaticity component in the water to be treated. In the example of FIG. 3, only one compartment is included, and the reaction tank 92 in which only one air diffuser plate 32 of the aeration equipment is disposed is illustrated. However, the reaction tank 92 may have a plurality of compartments, each of which has the air diffuser plate 32 disposed therein, as in the reaction tank 12 of the first embodiment described above.

Note that in the configuration of the second embodiment, the carrier immersion tank 71 and the reaction tank 92 are included in a treatment mechanism for treating wastewater by Bacillus proteolyticus. The immobilized carrier 81 immersed in the carrier immersion tank 71 may be included in the treatment mechanism. In addition, the aeration equipment (blower 31, air supply pipe 41, valve 51, and air diffuser plate 32) and return equipment (return pipe 42 and pump 21) may be included in the treatment mechanism.

In addition, although not illustrated in FIG. 3, similarly to the sludge discharge pipe 43 and the pump 22 of the first embodiment, equipment for removing excess sludge settled at the bottom of the final settling basin 13 may be connected to the return pipe 42 of the return equipment.

The wastewater treatment apparatus 200 of the second embodiment includes a controller 62. The controller 62 is configured as a computer including, for example, a CPU, ROM, RAM, etc., similarly to the controller 60 of the first embodiment described above, and controls each part of the wastewater treatment apparatus 200, for example, including temperature adjustment and heat retention of the carrier immersion tank 71.

According to the wastewater treatment apparatus 200 of the second modification, similar effect as that of the wastewater treatment apparatus 100 of the first embodiment is obtained.

According to the wastewater treatment apparatus 200 of the second embodiment, the carrier immersion tank 71 disposed on the upstream side of the reaction tank 92 is provided. In this way, the immobilized carrier 81 on which Bacillus proteolyticus is immobilized can be used for treatment of the water to be treated. By using the immobilized carrier 81, Bacillus proteolyticus can be maintained in the biological treatment system. Further, by appropriately replenishing the carrier immersion tank 71 with the immobilized carrier 81, it is possible to suppress changes over time (aging) such as deterioration of processing performance.

### (Modification)

Next, a wastewater treatment apparatus 201 of a modification of the second embodiment will be described with reference to FIG. 4. The wastewater treatment apparatus 201 of the modification is different from the wastewater treatment apparatus 200 of the second embodiment in that a rotating disc 82 is used as the immobilized carrier.

FIG. 4 is a diagram illustrating an example of a configuration of the wastewater treatment apparatus 201 according to the modification of the second embodiment. In FIG. 4, the same reference symbols are given to the same configurations as those of the above-described second embodiment, and a description thereof will be omitted.

As illustrated in FIG. 4, the wastewater treatment apparatus 201 of the modification includes a carrier immersion tank 72 capable of immersing the rotating disc 82 instead of the carrier immersion tank 71 of the second embodiment. For example, the carrier immersion tank 72 may include a heater (not illustrated) that heats the inside of the carrier immersion tank 72 to a temperature of about 30° C. suitable for the activity of Bacillus proteolyticus.

The rotating disc 82 is a carrier constituting an immobilized carrier that dominantly attaches and immobilizes Bacillus proteolyticus as a disc-shaped rotating body. In order to immobilize Bacillus proteolyticus, for example, a fibrous contact body is arranged on a surface of the rotating disc 82.

By rotating the rotating disc 82 using power of a motor (not illustrated) etc. in a state where at least a part of the rotating disc 82 is immersed in the carrier immersion tank 72, the entire surface of the rotating disc 82 can be brought into contact with the water to be treated in the carrier immersion tank 72.

In the carrier immersion tank 72, the enzyme produced by Bacillus proteolyticus is eluted in the water to be treated in contact with the rotating disc 82. The water to be treated is sent to the reaction tank 92 in a subsequent stage in a state where the enzyme having this pigment decomposition ability is contained.

Note that in the configuration of the modification, the carrier immersion tank 72 and the reaction tank 92 are included in a treatment mechanism for treating wastewater by Bacillus proteolyticus. The rotating disc 82 immersed in the carrier immersion tank 72 may be included in the treatment mechanism. In addition, the aeration equipment (blower 31, air supply pipe 41, valve 51, and air diffuser plate 32) and return equipment (return pipe 42 and pump 21) may be included in the treatment mechanism.

The wastewater treatment apparatus 201 of the modification includes a controller 63. The controller 63 is configured as a computer including, for example, a CPU, ROM, RAM, etc., similarly to the controller 60 of the first embodiment described above, and controls each part of the wastewater treatment apparatus 201, for example, including temperature adjustment and temperature retention of the carrier immersion tank 72 and rotation control of the rotating disc 82.

According to the wastewater treatment apparatus 201 of the modification, similar effect as that of the wastewater treatment apparatus 200 of the second embodiment is obtained.

According to the wastewater treatment apparatus 201 of the modification, the rotating disc 82 in which Bacillus proteolyticus is dominantly immobilized is disposed on the upstream side of the reaction tank 92. In this way, by immobilizing and dominating Bacillus proteolyticus on the rotating disc 82, the pigment component can be decomposed more efficiently. In addition, it is possible to save space.

Note that in the second embodiment and the modification, the carrier immersion tanks 71 and 72 capable of immersing the immobilized carrier 81 or the rotating disc 82 are included. However, in an apparatus configuration such as the wastewater treatment apparatus 100 of the first embodiment described above, the immobilized carrier 81 or the rotating disc 82 may be directly immersed in the reaction tank 12.

### Examples

Next, examples will be described with reference to the drawings. In the examples, the decomposition ability of the organic chromaticity component of the enzyme produced by Bacillus proteolyticus was evaluated. In the following experiment, Bacillus proteolyticus was obtained by isolating from a sludge.

(Culturing of Bacillus proteolyticus)

Bacillus proteolyticus was inoculated into a liquid culture medium containing components shown in Table 1 and cultured at 30° C. for 72 hours to multiply Bacillus proteolyticus.

TABLE 1

| Component | Content |
|---|---|
| Acid Red88 | 0.02 g (20 ppm) |
| Nutrient broth | 8 g |
| Glucose | 8 g |
| Sodium chloride | 6 g |
| Soluble starch | 10 g |

By containing Acid Red 88 which is an azo dye in the liquid culture medium, it is possible to suppress the multiplication of microorganisms other than Bacillus proteolyticus.

### (Decomposition and Removal Test of Organic Chromaticity Component)

A decomposition and removal test of the organic chromaticity component was performed using Bacillus proteolyticus cultured as described above. Acid Red 88 was used as an example of a persistent organic chromaticity component. That is, the culture solution containing cultured Bacillus proteolyticus was added to the liquid culture medium containing Acid Red 88, which has the same composition as shown in Table 1 and larger liquid volume. The removal performance of Acid Red 88 by Bacillus proteolyticus was evaluated while shaking and culturing at a temperature of about 30° C.

The removal performance of Acid Red 88 was evaluated by chromaticity measurement. For the chromaticity measurement, a method of measuring the absorbance of the liquid culture medium containing Acid Red 88 with respect to light having a wavelength of 504 nm was used. The higher the absorbance for light having the wavelength of 504 nm, the stronger red color of the liquid culture medium, that is, the higher content of Acid Red 88.

In addition, a removal rate of Acid Red 88, which is an organic chromaticity component, was determined from the measured absorbance. More specifically, an absorbance of a standard sample having a known pigment concentration prepared in advance was measured at a plurality of concentrations to obtain a calibration curve formula. The absorbance of the liquid culture medium containing Bacillus proteolyticus measured as described above was substituted into this calibration curve formula to calculate the pigment concentration of the liquid culture medium. The removal rate of Acid Red 88 was calculated based on the pigment concentration EV obtained by calculation of the liquid culture medium after the lapse of a predetermined time with reference to the pigment concentration IV obtained by calculation of the liquid culture medium immediately after addition of the culture solution containing Bacillus proteolyticus. A calculation formula for obtaining the removal rate from the pigment concentrations IV and EV is shown below.

Removal rate (%)=100×(IV−EV)/IV

As the decomposition of Acid Red 88 in the liquid culture medium progresses over time, the absorbance and pigment concentration decrease due to the decrease in the content of Acid Red 88. Therefore, when the removal rate increases over time, this increase indicates that the decomposition of Acid Red 88 is in progress.

### (Changes in Absorbance Over Time)

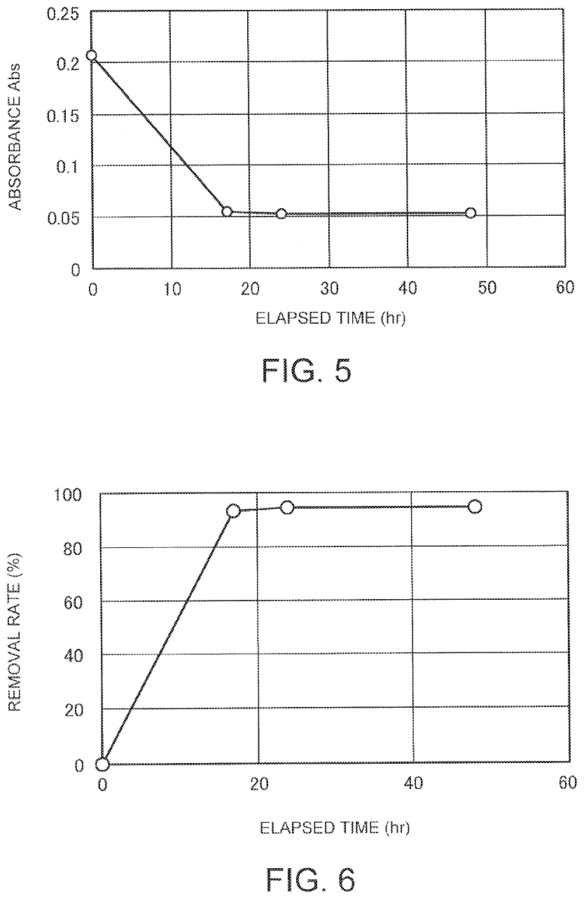

FIG. 5 is a graph illustrating a change over time in an absorbance of an Acid Red 88-containing liquid culture medium to which a Bacillus proteolyticus-containing culture solution is added according to the example. In the graph of FIG. 5, a horizontal axis is the elapsed time (hr), and a vertical axis is the absorbance (Abs) with respect to light having a wavelength of 504 nm in the liquid culture medium containing Acid Red 88.

As illustrated in FIG. 5, the absorbance of the liquid culture medium was more than 0.2 immediately after the addition of the culture solution containing Bacillus proteolyticus, that is, when the elapsed time was 0 hours. On the other hand, 17 hours after the addition of the culture solution containing Bacillus proteolyticus, the absorbance of the liquid culture medium decreased to about 0.05. From this result, it can be seen that Acid Red 88 in the liquid culture medium was decomposed by the culture solution containing Bacillus proteolyticus.

### (Change in Removal Rate Over Time)

FIG. 6 is a graph illustrating a change over time in a removal rate of Acid Red 88 contained in a liquid culture medium to which Bacillus proteolyticus-containing culture solution is added according to the example. In the graph of FIG. 6, a horizontal is the elapsed time (hr), and a vertical axis is the decrease rate (%) of Acid Red 88 contained in the liquid culture medium.

As illustrated in FIG. 6, 17 hours after the addition of the culture solution containing Bacillus proteolyticus, the removal rate of Acid Red 88 contained in the liquid culture medium was 93%. In other words, it can be seen that of Acid Red 88 initially contained in the liquid culture medium, 93% of the Acid Red 88 was removed after 17 hours.

Even though some embodiments of the invention have been described, these embodiments are presented as examples and are not intended to limit the scope of the invention. These novel embodiments can be implemented in various other modes, and various omissions, replacements, and changes can be made without departing from the gist of the invention. These embodiments and modifications thereof are included in the scope and gist of the invention, and are included in the scope of the invention described in the claims and the equivalent scope thereof.

What is claimed is:

1. A wastewater treatment apparatus, comprising:
a treatment mechanism that treats a wastewater containing an organic chromaticity component with an enzyme produced by Bacillus proteolyticus.

2. The wastewater treatment apparatus according to claim 1,
wherein the enzyme reduces a chromaticity of the wastewater.

3. The wastewater treatment apparatus according to claim 1,
wherein the organic chromaticity component is an azo dye, and
the enzyme decomposes at least a part of an azo bond contained in the azo dye.

4. The wastewater treatment apparatus according to claim 1,
wherein the treatment mechanism includes a reaction tank for causing a reaction between the enzyme and the organic chromaticity component in the wastewater.

5. The wastewater treatment apparatus according to claim 4,
wherein the reaction tank is configured to be able to bring the wastewater into contact with at least one of the Bacillus proteolyticus, an immobilized carrier on which the Bacillus proteolyticus is immobilized, and a rotating disc configured to be able to rotate the immobilized carrier.

6. The wastewater treatment apparatus according to claim 4,
wherein the treatment mechanism further includes a culture tank connected to the reaction tank, and
the culture tank is configured to be able to multiply the Bacillus proteolyticus, and supplies a culture solution containing the multiplied Bacillus proteolyticus to the reaction tank.

7. The wastewater treatment apparatus according to claim 6, further comprising
a controller that controls at least one of a supply timing and an amount of supply of a culture medium containing the Bacillus proteolyticus from the culture tank to the reaction tank.

8. The wastewater treatment apparatus according to claim 7,
wherein the controller controls at least one of the supply timing and the amount of supply according to a concentration of the organic chromaticity component in the wastewater.

9. The wastewater treatment apparatus according to claim 4,
wherein the treatment mechanism further includes a culture solution tank connected to the reaction tank, and
the culture solution tank is configured to be able to store a supernatant liquid after separating the Bacillus proteolyticus from a culture solution in which the Bacillus proteolyticus is cultured or a concentrated liquid obtained by concentrating the supernatant liquid, and supplies the stored supernatant liquid or the concentrated liquid to the reaction tank.

10. The wastewater treatment apparatus according to claim 9, further comprising
a controller that controls at least one of a supply timing and an amount of supply of the supernatant liquid or the concentrated liquid from the culture solution tank to the reaction tank.

11. The wastewater treatment apparatus according to claim 10,
wherein the controller controls at least one of the supply timing and the amount of supply according to a concentration of the organic chromaticity component in the wastewater.

12. The wastewater treatment apparatus according to claim 4,
wherein the treatment mechanism further includes a microbial product container connected to the reaction tank, and
the microbial product container is configured to be able to contain a microbial product containing the Bacillus proteolyticus, and supplies the microbial product to the reaction tank.

13. The wastewater treatment apparatus according to claim 12,
wherein a form of the microbial product is a powder form, a liquid form, or a tablet form.

14. The wastewater treatment apparatus according to claim 13, further comprising
a controller that controls at least one of a supply timing and an amount of supply of the microbial product from the microbial product container to the reaction tank.

15. The wastewater treatment apparatus according to claim 14,
wherein the controller controls at least one of the supply timing and the amount of supply according to a concentration of the organic chromaticity component in the wastewater.

16. The wastewater treatment apparatus according to claim 1,
wherein the treatment mechanism includes
a reaction tank that causes a reaction between the enzyme and the organic chromaticity component in the wastewater, and
a carrier immersion tank disposed on an upstream side of the reaction tank, and
the carrier immersion tank is configured to be able to immerse an immobilized carrier on which the Bacillus proteolyticus is immobilized.

17. The wastewater treatment apparatus according to claim 16,
wherein the immobilized carrier is a fluidized bed carrier, and
the fluidized bed carrier flows in the carrier immersion tank by the wastewater flowing into the carrier immersion tank.

18. The wastewater treatment apparatus according to claim 16,
wherein the immobilized carrier is a rotating disc allowed to rotate while being in contact with the wastewater.

19. A wastewater treatment method, comprising:
treating the wastewater that contains an organic chromaticity component with an enzyme produced by Bacillus proteolyticus.

20. The wastewater treatment method according to claim 19,
wherein the enzyme reduces a chromaticity of the wastewater.

21. The wastewater treatment method according to claim 19,
wherein the organic chromaticity component is an azo dye, and
the enzyme decomposes at least a part of an azo bond contained in the azo dye.

22. The wastewater treatment method according to claim 19,
wherein the wastewater is brought into contact with at least one of a culture solution containing the Bacillus proteolyticus, a supernatant liquid after separating the Bacillus proteolyticus from the culture solution, a concentrate liquid obtained by concentrating the supernatant liquid, a microbial product containing the Bacillus proteolyticus, an immobilized carrier on which the Bacillus proteolyticus is immobilized, and a rotating disc configured to be able to rotate the immobilized carrier.
